(19) United States
(12) Patent Application Publication
(10) Pub. No.: US 2018/0130373 A1
(43) Pub. Date: May 10, 2018

(54) EXERCISE MANGEMENT SYSTEM WITH BODY SENSOR

(71) Applicants: Corinne Bernard-Paroly, Harrison, NY (US); Keith Connelly, Ithaca, NY (US)

(72) Inventors: Corinne Bernard-Paroly, Harrison, NY (US); Keith Connelly, Ithaca, NY (US)

(21) Appl. No.: 15/809,586

(22) Filed: Nov. 10, 2017

Related U.S. Application Data
(63) Continuation-in-part of application No. 14/996,741, filed on Jan. 15, 2016, now abandoned.
Application No. 14/996,741 is a continuation-in-part of application No. 14/091,571, filed on Nov. 27, 2013, now abandoned.
(60) Provisional application No. 62/428,598, filed on Dec. 1, 2016.
(60) Provisional application No. 61/731,022, filed on Nov. 29, 2012.

(51) Int. Cl. G09B 19/00; G09B 5/06; A63B 69/00; G16H 20/30

(52) U.S. Cl. CPC G09B 19/0038; G09B 5/065; A63B 69/00; G06F 17/30817; A63B 2220/40; A63B 2220/803; A63B 2220/836; G16H 20/30

(57) ABSTRACT

This invention pertains to a system for providing exercise programs to users based on regimens prepared by health care practitioners. Each regimen is automatically converted into a set of exercise sessions, each session consisting of one or more exercises. The exercise sessions are provided to users as audio/video programs. Preferably, an avatar in the video portion of each program performs the respective exercises and together with the audio portion provides prompts, encourages and instructs the user in following and performing the exercises. In addition, a plurality of sensors may be deployed for concurrently receiving and processing motion data for display and analysis relative to exercise performance.

### CROSS-REFERENCE TO RELATED APPLICATION

This patent application claims benefit under 35 U.S.C. §119 to U.S. Provisional Patent Application No. 62/428,598, filed Dec. 9, 2016, is a continuation-in-part of U.S. patent application Ser. No. 14/996,741, filed on Jan. 15, 2016, which is a continuation-in-part of U.S. patent application Ser. No. 14/091,571, filed Nov. 27, 2013, which claims priority to U.S. Provisional Patent Application No. 61/731,022, filed Nov. 29, 2012, each incorporated herein by reference in its entirety.

### FIELD OF THE INVENTION

This application pertains to a system and method of generating and presenting an exercise regimen to a person that may be based on a prescription by a health professional. The regimen is presented to the person on a display, potentially including audio, such as by an avatar animated using a game engine.

### BACKGROUND OF THE INVENTION

Exercising regimens are provided for many people for many reasons, such as a means for promoting recovery after some illness, losing weight, maintaining the person's physical shape, training for an athletic event, and so on. Regimens are typically prescribed by doctors or other health-related professionals, such as physical or occupational therapists, or they can be self-imposed. There are also various audio/visual materials available for demonstrating how to perform exercises. However, these materials are generally generic and cannot be customized to the needs of a particular person.

It is well known that people find in it much easier to perform exercises, especially consisting of long, tedious regimens, if they could share the activity or do the same activity with another person. That is one of the reasons why gyms and other venues were several people can exercise together have become so popular. However, because each person, especially a person recovering from an illness, has his or her personal needs and requirements, existing exercising sessions at a gym.

### SUMMARY OF THE INVENTION

Briefly, this application pertains to a system for providing exercise programs to users based on regimes prepared by health care practitioners. Each regime is automatically converted into a set of exercise sessions with each session consisting of one or more exercises. The exercise sessions are provided to users as audio/video programs. In each routine, an avatar performs the respective exercises as the video portion, while the audio portion provides prompts, encourages and instructs the user in following and performing the exercises.

In order to make the system user friendly, each avatar is custom designed for the users. In addition, elements in each program, including background images, background music, the appearance of avatars, and so on, can be changed from session to session.

In an embodiment, the movements of the avatars are generated and/or otherwise implemented using a game engine. In other words, each exercise for a given exercise session consists of a series of movements for various body parts. These series of movements are provided to the game engine which uses the motion capture animation of each exercise to generate the video portion of each routine, so that the avatar performs the specific routine that follows the exact prescription for each patient.

In an embodiment, a server is provided that receives the regimes and uses libraries of motion capture animations of each exercise to generate the video portion of the routine as performed by an avatar, along with the other elements required for each routine. During each exercise session, the user is shown not only the avatar video portion and the technique instruction audio, but also may include various information including but not limited to historic data related to his or her performance of the exercise sessions.

In an embodiment, body sensors are used to detect movement of a user's body to confirm that the user is matching the movements of the avatar, and/or to generate a second avatar of the user, based on information from the sensors.

### BRIEF DESCRIPTION OF THE DRAWINGS

FIG. 1 is a block diagram of a system for providing exercise programs for a user in accordance with this invention;

FIG. 2 is a block diagram of the server of FIG. 1;

FIG. 3A is a flow chart illustrating the process used by the server for generating the exercise program;

FIG. 3B is a flow chart for describing how the program generates the sequence of exercises within a routine;

FIG. 3C is a flow chart of how the server delivers an exercise session on demand from a user;

FIG. 3D is a flowchart of how the server updates its information at the end of an exercise session;

FIG. 4 is a sequence of oral and visual segments that make of a typical exercise session;

FIG. 5 is a somewhat diagrammatic representation of how the respective avatar and related information is shown to a user during an exercise session;

FIG. 6 is a block diagram of a user device used to obtain an exercise program from the server;

FIG. 7 is a flow chart of the operation of the device of FIG. 6; and

FIG. 8 is a diagram of a body with a plurality of body sensors used to generate videos of a user during exercise.

### DETAILED DESCRIPTION OF EMBODIMENTS OF THE INVENTION

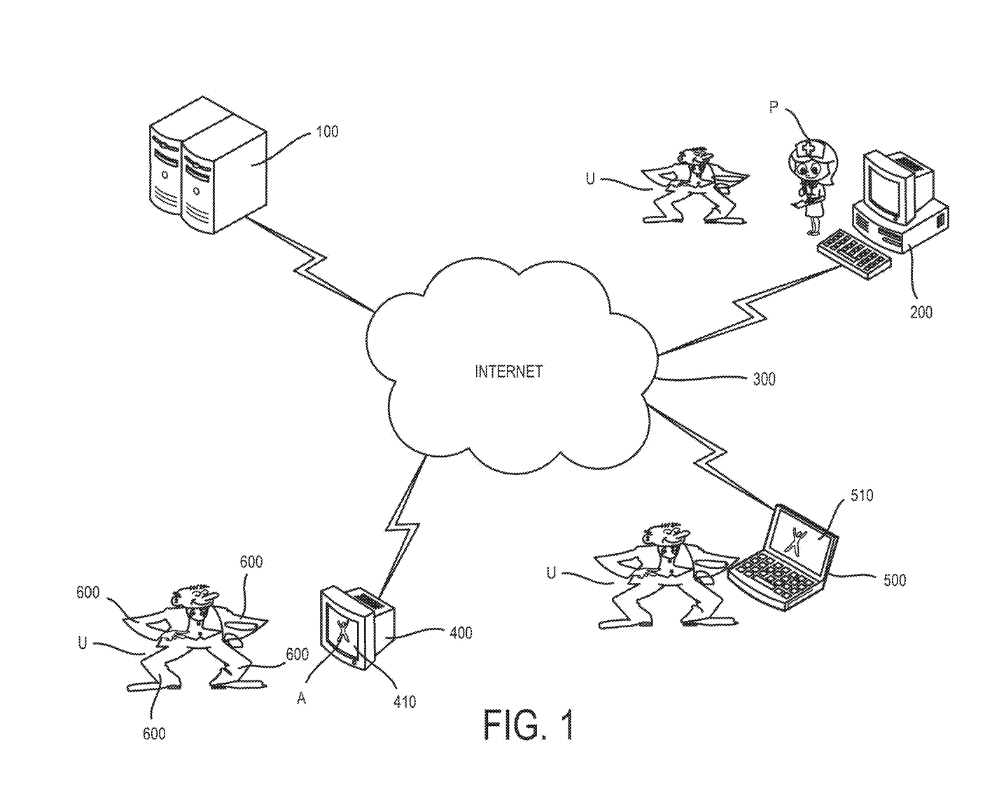

In a typical scenario, as shown in FIG. 1, a user U visits a health care practitioner P who examiners the user U and the medical records of the user U and based on various factors, describes a regimen R of exercises. This regimen R normally consists of several exercises. The practitioner P may be a doctor, a physical or occupational therapist, a trainer, etc.

The practitioner P then, using a computer 200 interacts with an exercise program server 100 through the Internet 300 to generate an exercise program EP based on the prescribed regimen R. The regimen R may be generated by the practitioner P electronically on the device 200. In one embodiment, the practitioner P generates the regimen R by first accessing a website associated with the server 100 and then filling out the information defining the prescribed regimen R. If the user U is an old patient, the practitioner P also associates the exercise program EP with the profile of the user U. If user U is a new patient, the practitioner P generates a profile for the user U and provides other information including avatar information needed to generate a personalized avatar for the user as well as the exercise program EP. This information is then used by the server 100 to generate the appropriate exercise program ER. As discussed above, the regimen could also be created by a health and exercise trainer working with user U or even by the user U.

Alternatively, the practitioner P generates the regimen R manually (In this latter case, the regimen may be transferred to the server in an electronic version at a later time).

The routine R typically requires that the user U perform the routine R over a period of time, e.g., several months, to be performed one or more.

After the user U visited the practitioner P, he can access the specific exercise routine R tailored for him, through the internet 300 and a permanent user device 400. Device 400 may be located for example, in the user's home. The device 400 may be associated with a data storage 410. In one embodiment, the server 100 transmits the whole exercise program to the user device 400 which is then stored in the data storage 410 and the user is then free to interface with the routine R at will, without any further information required from the server 100. In an alternate embodiment, the individual exercise session ES may be provided one at a time on demand and may be customized as necessary. As will be described in more detail below, each of the sessions ES are stored in the data storage 410 and the user then can access either the latest ES or any of previously received sessions. Alternatively, for example, if he is travelling and is using a temporary access device 500, instead of providing the whole program EP, the server 100 provides only one of the sessions ES or another subset to the user. For example, the user U may be using a laptop as the temporary access device 500 in a hotel room.

The permanent device 400 is preferably and primarily used on a desktop or laptop based device, although it could also or alternatively be incorporated into or implemented on a tablet, or even a hand-held device. In any event, the user can initiate an exercising session at any time or place where and when he is ready for it. Thus the device 400 can be used in an office, at home, on the road in a hotel room, a health spa, etc.

As mentioned above, the device 400 may receive the whole program EP at once, but preferably receives each session or portions of each session separately. More specifically, when the user U is ready for a session, he signs in on server 100 from his device 400 (or 500) (preferably through the internet 300) that he is ready for a session.

Preferably, data D is generated by device 400 each time user U is viewing (and presumably performing) an exercise session ES. This data D, includes the time and date the viewing has occurred. This data D is sent back for tracking user U to the practitioner and the server 100.

One important feature of the invention is that during each exercising session, the user U is prompted to do each of the particular exercises designated by the respective session. The device 410 includes a display 414 and one or more speakers 416. Preferably, the prompting is done audio visually by presenting on display 414 an avatar A and playing sounds through one or more speakers 416. (Temporary device 500 has similar displays and speakers, not shown).

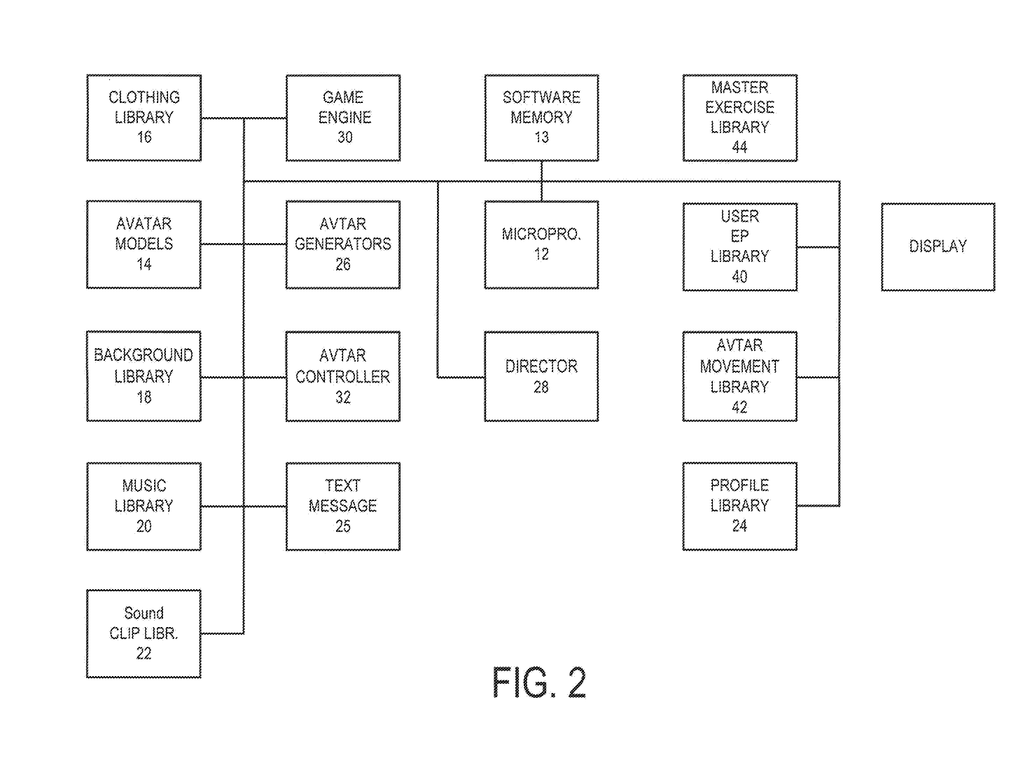

The process for generating the exercise program EP and its components, the exercise sessions ES is now described. As shown in FIG. 2, the server 100 includes a microprocessor 12 and several modules that may be implemented by software running on the microprocessor 12 (and stored in a memory 13) however individual modules are illustrated herein for the sake of clarity.

The avatar A may be an anatomically correct (preferably human) figure with a head, a body and arms and legs that move to simulate each particular exercise. The avatar could be a generic figure, however, preferably the image of the avatar is customized in order to provide more realism, enhance the quality of the experience, and recreate an environment and experience for the user U (or more than one users) that simulates a sharing between several people or the experience of exercising under the direction of a trainer. For this purpose, a plurality of models for avatars are provided in a model library 14. The images may include models having different characteristics such, height, weight, skin color, hair color, haircut, etc. In one embodiment, images may be of well-known real or imaginary persons. A clothing library 16 holds images of various articles of clothing to be worn by the avatar. Various colors for the articles of clothing, including well known insignia or brand names may also be included in the clothing library. A background library 18 provides various backgrounds against which the image of the avatar A is presented.

A music library 20 holds various songs and a sound clip library 22 holds sound clips of instructions and other sounds.

A text message library 25 holds text messages.

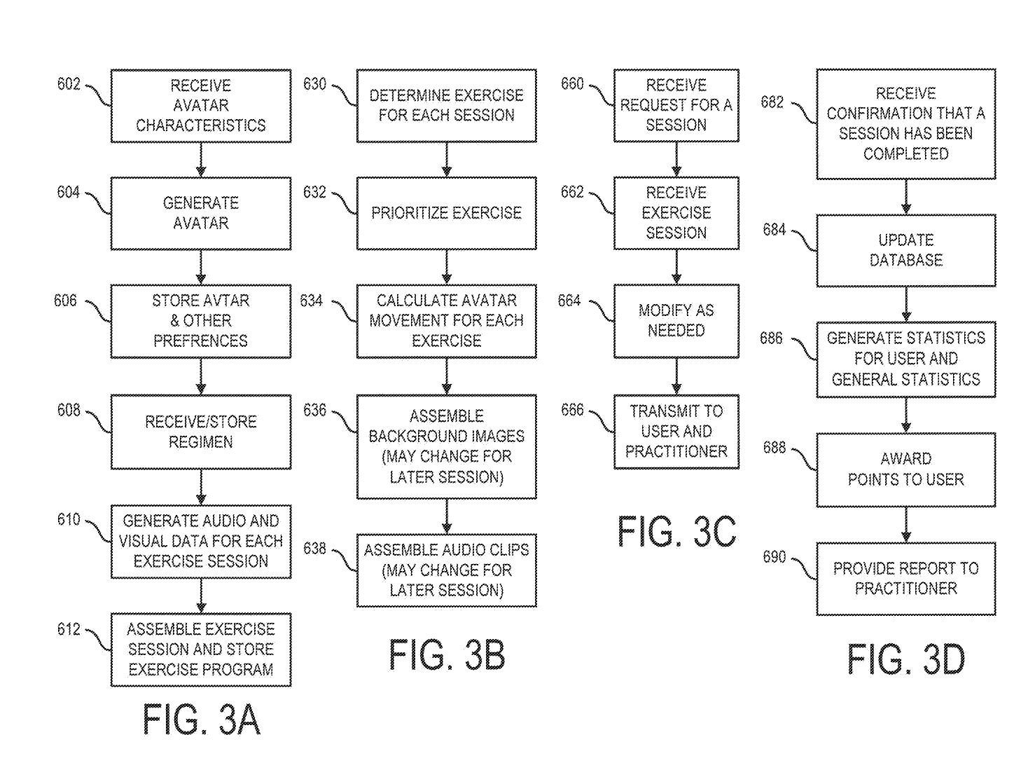

When the user U is signed up to the system, a preconstructed avatar is either assigned to him or a new avatar is created for him either by practitioner P or some other personnel. For example, as shown in the flow chart of FIG. 3A, in step 600, the desired avatar characteristics are provided to avatar generator 26 and based on these characteristics a suitable avatar image is selected from the avatar models 14. For example, if the user U is a female, she may prefer to view an avatar that is also female. In addition, clothing articles and colors are also chosen for the avatar from clothing library 16. All these characteristics may be constant for all the exercise sessions, or they may change from session to session (or at will) to add to the realistic effects. For example, at the beginning of an exercise session the user may send a message to the server that she wants to change the clothes on the avatar assigned to her. The avatar generator 26 performs the necessary changes and generates a new avatar for the user to be used for that exercise session or a future session (step 604). Once the avatar for the user is constructed by generator 26, in step 602, the avatar and other parameters and information associated with a specific user U (including his musical preferences, background preferences, etc.) are stored in the profile library 24 (step 606). If the user has a profile already, it is retrieved from the profile library 24 as needed.

A master exercise library 44 is used to store all the exercises that the server 100 can incorporate into an exercise program EP.

A regimen prescribed by the practitioner P is received by the server 100 and stored in the profile library for each user U (step 608). Next, in step 610, the regimen for user U is used to generate the corresponding exercise program EP with its component exercise sessions ES is generated and stored in EP library 26. Each exercise called for by regimen R may be retreated from the library 44.

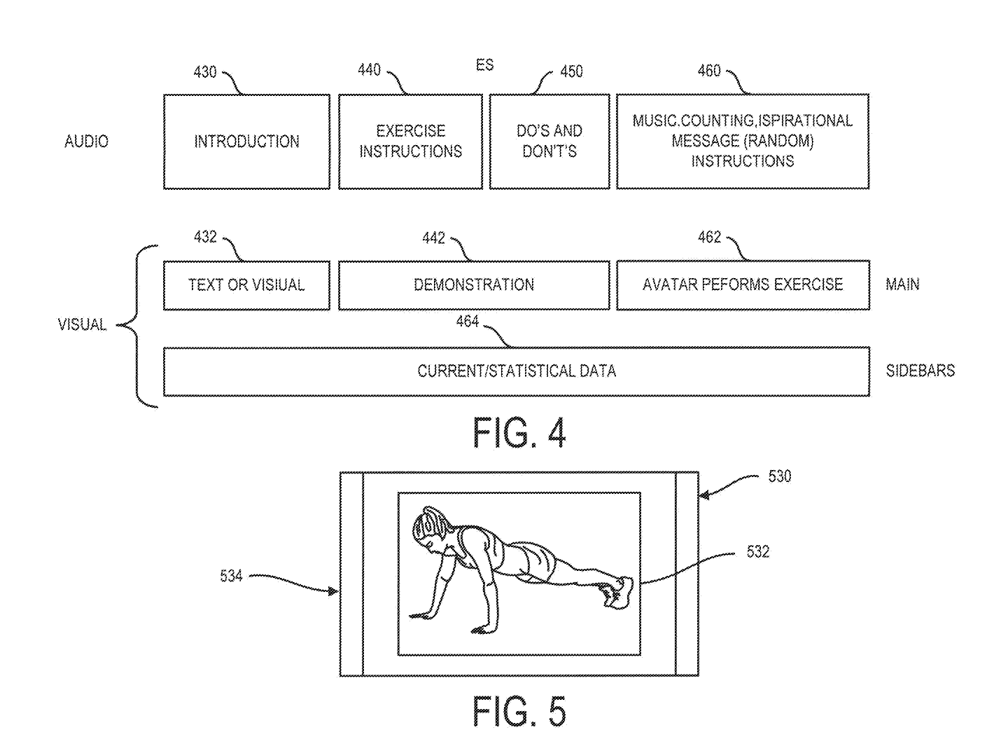

A typical exercise session, as shown in FIG. 4 includes an audio and a visual component, each component preferably consisting of respective segments. Preferably, the visual component includes a main section which is generally shown at the center of the displays 410, 510 and one or more sidebars that are arranged around the perimeter of the displays. More specifically, as shown in FIG. 5, a composite image 530 shown during an exercise session ES may include a main section 532 showing avatar A (with some optional background images that have been omitted for the sake of clarity) and one or two side bars 534.

Referring back to FIG. 4, each session ES starts off with an introduction segment 430, in which the user U is provided with general information about the session. During this time, text or other visual images are shown in main section 552 (segment 432). Next, exercise instructions 440 are provided, preferably accompanied by a visual demonstration segment 442 in which the avatar demonstrates the exercises. Technique instructions are provided both audibly and visually (segments 450, 442). Finally, the actual exercise segments 460, 462 are played. During these segments, the avatar is shown performing the respective exercises, while the audio track plays a real time count synchronized with the movement of the avatar. So, for example, if an exercise session includes ten pushups, the avatar may perform pushups and each pushup may be counted out loud in the sound track. Additional sound clips are also played either in the background (e.g., music) or other messages are played between the counts, including inspirational messages (that may be played at random), additional instructions, and so forth.

The session ES can be paused and resumed at any time.

Moreover, some of the instructions and messages can be shortened, or eliminated after they have been played several times. For example, if the same sessions ES is being played for the fifth time, the introduction can be eliminated, together with all or some of the exercise instructions or do's and don'ts. The speed at which the exercise is performed can also be changed automatically. For example, initially, the avatar can be performing pushups at a low rate, e.g., 1 per second. If the user is playing the same session ES the fourth time in a week, the rate may be increased to four pushups in three seconds. After 10 sessions, the rate may be increased to 2 pushups per second. Moreover, each movement in any given exercise can have its time and speed specified for any user. The user and/or practitioner can optionally change this rate manually. A professional can change the angle similarly to changing the speed. As a patient progresses, their injured body part should display an improved range of motion over time and the angle can be increased to match it. As discussed above, long and short term changes may be made as well. For example, the background images can be changed with each season, the colors of the clothing form the avatar may be changed, the music played may be changed, the music may be changed every time the session is played, etc. In this manner, the user can view (and hopefully participate with) the same exercise session several times and each time the experience will be slightly different to make it a more entertaining and exciting experience for the user. Preferably, these changes are made by the server before the respective session ES is downloaded.

The sidebar 534 is used to provide additional information during each session, such as the time when the session was started, how long is the session, how much time has elapsed since the session has started, how much is left in the session, how many times an exercise has been repeated, what is the next exercise within the session, what exercises are included in the next session, how many times the user has watched this current session, etc.

Returning to FIGS. 2 and 3A, after a regimen is stored in step 608, a director 28 analyzes the regimen, collects the required background images, sound clips, text messages, and puts together the audio and visual tracks for each of the exercise session (illustrated in FIGS. 4 and 5, step 610 in FIG. 3A) of the program, and stores them in the user EP library 40 (step 612).

Importantly an avatar movement library 42 is provided which defines a series of movements required to be performed by the avatar A for each exercise. For example, for each pushups, the arms, legs, torso, head and neck of the avatar have to perform certain precise movements. A game engine 30 and an avatar controller use the information from the library 42 to generate video images of the respective avatar performing the respective exercise in real time. This information becomes part of the respective ES and is stored with all the other information into user EP library 40. Moreover, even for the same ES, some details of the exercise, e.g., the rate and angle at which an exercise is performed by the avatar is changed either automatically or in response to a request/command from user U.

In one embodiment, the game engine 30 and the director 28 are placed in the user device 400, 500 and the renderings for avatar's motions are generated there rather than in the server.

FIG. 3B shows more details for step 612. More specifically, many exercise sessions consist of a plurality of exercises with the user taking several positions for each exercise and which different exercising addressing different muscles, tissues, joints, etc. For example, one exercise session could include a first set of exercises with the user standing, a second set of exercises with the user lying on his left side and a third set of exercises while the user is lying on his right side. Another exercising session includes a first set of exercises requiring a set of exercising involving a first equipment (e.g., a foam roller), a second set of exercises involving a second equipment (e.g., weight lifting) and so on. The director 28 reviews and categorizes the exercises within each exercise session based on a predetermined criteria, e.g., user position, equipment used, etc. (Step 630). Next, the director 28 prioritizes and orders the exercises in a sequence using predetermined rules. For example, the director may order the exercises so that all the exercises on the left side are done, followed by all the exercises on the right side, all the exercises, requiring standing, etc. Alternatively, the director 28 may put all the exercising requiring a chin bar followed by exercising requiring weights, etc. In another alternative embodiment the director 28 may group all the exercises together that are directed at strengthening the arms, followed by exercises for the leg, etc. In yet another embodiment, the director 28 may order the exercises in a manner that does not tire out certain members. So for example, exercises for the arm may be interleaved with exercises for the leg.

Once the sequence of exercises is set by director 28, in step 634 the proper avatar movements are calculated for the sequence defined in step 632. Next, one or more background pictures are obtained (step 636), the appropriate songs and sound clips are added (step 638) and the resulting exercise session is stored in library 40. Again, some of the determinations in steps shown in FIG. 38 can be permed by the user devices 400, 500. This later configuration is especially desirable if the user desires to change some of the exercise session parameters.

As shown in FIG. 3C, after the exercise program for a user has been determined, the server 100 is ready for operation. Typically, in step 660 the server 100 receives a request for an exercise session ES within exercise program EP. The server 100 checks the progress of the user and retrieves the appropriate exercise session (step 662). In step 664 the exercise session ES modified, if needed. For example, the exercise program EP may have been initially set up in the summer but the request for the latest ES occurs in the winter. The clothing for the avatar and the background images are changed to reflect that it is winter and not summer.

Finally, in step 666 the requested exercise session is sent to the user U and the practitioner may receive a notification of this matter as well.

As shown in FIG. 3D, once a user finishes watching any exercise session, the server 100 is notified by device 400, 500. The notification may include additional information, such as where was the session viewed, how much of the session was viewed by the user, etc. (step 682). In step 684 statistics are generated regarding user U and any other user managed by the server 100 and this information is entered into the profile liberty 24 as well as other databases interested in such statistics.

Certain milestones can be set within the server and when a user reaches certain milestones, rewards may be awarded to the user (step 688). For example, the user may be rewarded with points if he completes the first N sessions in a predetermined time. Information is also sent to the practitioner P so that she can see what is going in with user U. For example, the practitioner may want to know if the user U has not finished some exercise sessions within a predetermined period of time, e.g., 3 weeks.

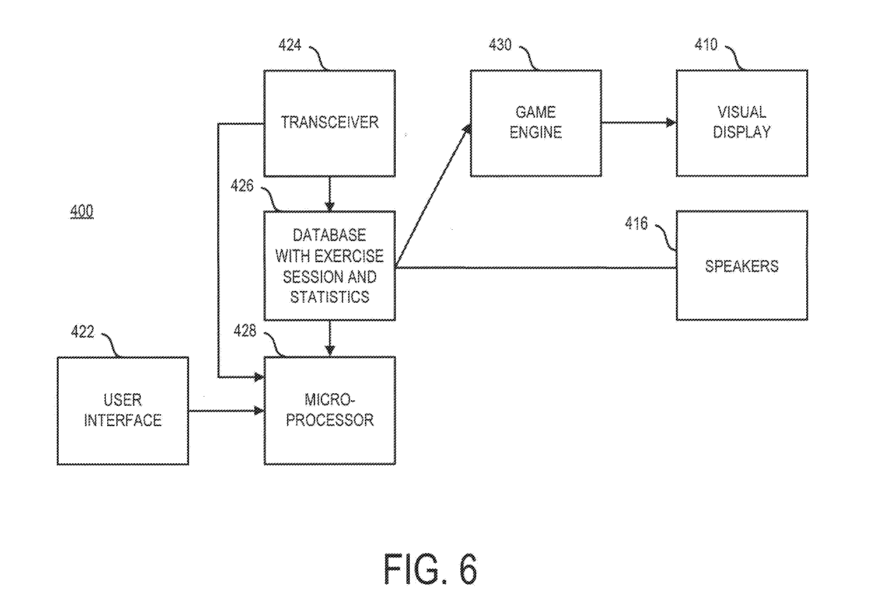

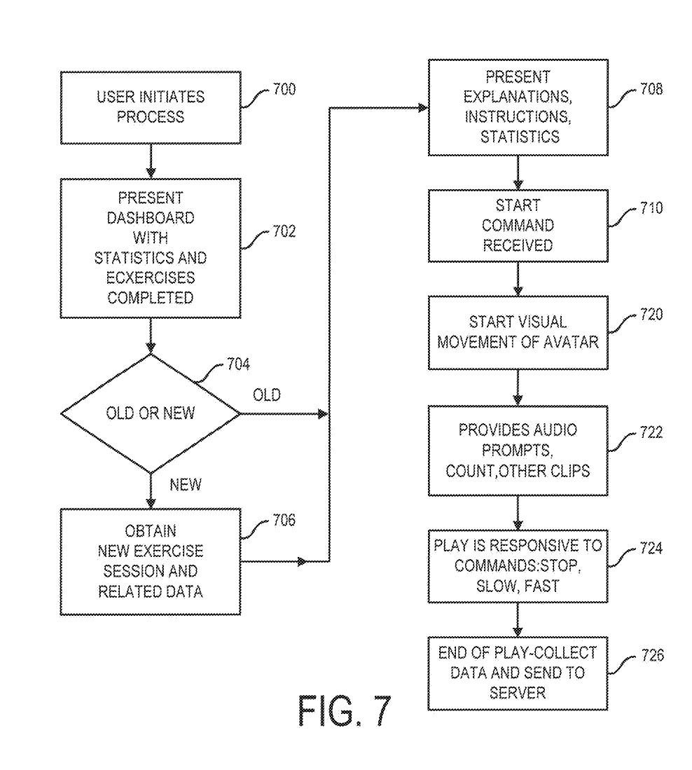

FIGS. 6 and 7 provide further detail of the operation of device 400. In addition to screen 410 and speakers 416, device 400 further includes a user interface 422 (such as a mouse, a keyboard, etc.), a transceiver 424 exchanging signals, normally with server 100, a database of downloaded exercise sessions and statistics associated with user U and his performance, and optionally, a game engine 430 as discussed above. When the user U is ready to exercise he initiates the process in step 700 (for example by activating an application on device 400. In step 702 a dashboard (not shown) is presented to the user U indicating various information and statistics, such as when was the last time he exercised, what was the latest ES he has viewed, etc.

The dashboard also includes icons for exercise sessions that have been previously downloaded into database 426 as well as the next ES that is due to be downloaded. In step 704 the user can select either one of the previously loaded ES or choose a new ES. If that is the user's choice, the latest ES is downloaded.

In step 708 the user is presented with the introduction, explanation, instructions, and statistics relevant to the user U. This step includes presenting to the user sections 430, 440, 450. In step 710 the user U initiates a start command indicating that he is ready to go on. In steps 720 and 722 the avatar A starts exercising with accompanying sound clips, prompts, count, etc. During play, the user U can issue commands requesting that the exercise session be terminated, stopped temporarily, slowed down, speeded up, cancelled, etc. (step 724). When the session is finished an appropriate command is sent to the server 10 and optionally to practitioner P to indicate the progress of user U, and other information, as discussed, by various conventional means, such as an email.

In one embodiment, the master exercise library contains a complete set of files of different exercises, each file being generated by recording a person performing the respective exercise using anywhere from one up to 200 or more cameras, digitizing the movements of the person (for example, by recording the movement of the person's limbs, joints, etc.) and recording the movement. The game engine may be used to generate a video of an avatar performing the same movement for the same exercise. Once this library is available, one or more practitioners P can choose a regime of exercises for one or more users, and the regime for each user can be customized for his or her needs, by choosing the exercises for each regime, the number of times it can be repeated, and/or variations in the repetition rate (e.g., a person can start with 5 sits ups and gradually go 15 sits up in 3 months), as examples. Moreover, the practitioner can also modify some of the exercises. So, for example, for a leg lift, the practitioner can chose a “hold period” of 0-10 seconds during which the position of the leg is maintained still up in the air.

Other parameters may be changed as well. For example, in one embodiment, the practitioner can prescribe whether during an exercise the avatar will hold an arm, a leg, etc., at a predetermined angle of let's say 30-75 degrees. Other parameters associated with specific exercises can be made variable as well. When the practitioner accesses a website associated with server 100, he can be presented with menus indicating the various exercises available, and for exercises with variable parameters, the practitioner can pick and choose the parameters (optionally with some suggested or recommended values for these parameters).

One important feature of the invention is that an exercise program may include a calendar option that tracks the days on which the user is expected to exercise, the exercise to be performed during each day (if any), etc. Each time the user U watches a respective exercise session, the system may assume that the user has performed the recommended exercises and the calendar is advanced accordingly (e.g., performing sit-ups 10 times the first week, 12 times the second week, etc.). The practitioner can receive real time feedback and can send a modification to the regime, if the practitioner P feels it is necessary.

One advantage of the invention is that during the generation of the data for each exercise, each movement can be performed by the live actor, but advantageously, duplicate movements may be eliminated. So, if for an exercise, an actor lifts his right leg and is recorded and then the movement for the left leg is required, the movement for the left leg need not be recorded but can be extrapolated for the avatar from the recording of the right leg. In other words, an exercise regime may require the lifting of the right leg 5 times followed by the lifting of the left leg. The director is configured to automatically obtain the data for the right leg once, extrapolate the movement for the left leg and the loop the data for the right leg first five times, followed by the extrapolated movement of the left leg repeated five times.

Moreover, as part of the sorting process described above, for example, for one session the right leg may be shown as moving first for the first week, and then the left leg is shown as moving first for the second week, and so on. In addition, in order to relieve the monotony, assuming for example, that a user must perform exercises five times a week, during sorting and arranging of the exercises, the avatar can be shown from different angles. So on Monday, in the first scene the avatar may be shown from the left side, on Tuesday from the left side, on Wednesday from the front, etc. For each subsequent scene on each day, the avatar could be shown from different angles as well. The angles are selected originally by the director to illustrate how an exercise is to be performed but can be changed dynamically from day to day, week to week, etc.

One feature of the invention is that each repetition for each exercise is counted out loud on the audio track may be used to encourage the user and so that the user does not have to do it himself. So, for example, if 15 pushups are required, the avatar takes the initial position for pushups, a “one” is heard on the audio track and the avatar performs the first pushup, then a “two” is heard on the audio track while the avatar is performing a second pushup and so on. Thus each repetition is tagged with a number and the number is announced concurrently with the execution of the exercise by the avatar (and hopefully the user). In this manner, each repetition is tagged with a number and the number is then announced as a count. Of course, the counting is properly synchronized with the speed at which the avatar is performing the exercise. If the avatar is performing pushups fast, the counting may be kept up so that it is announced at the same rate.

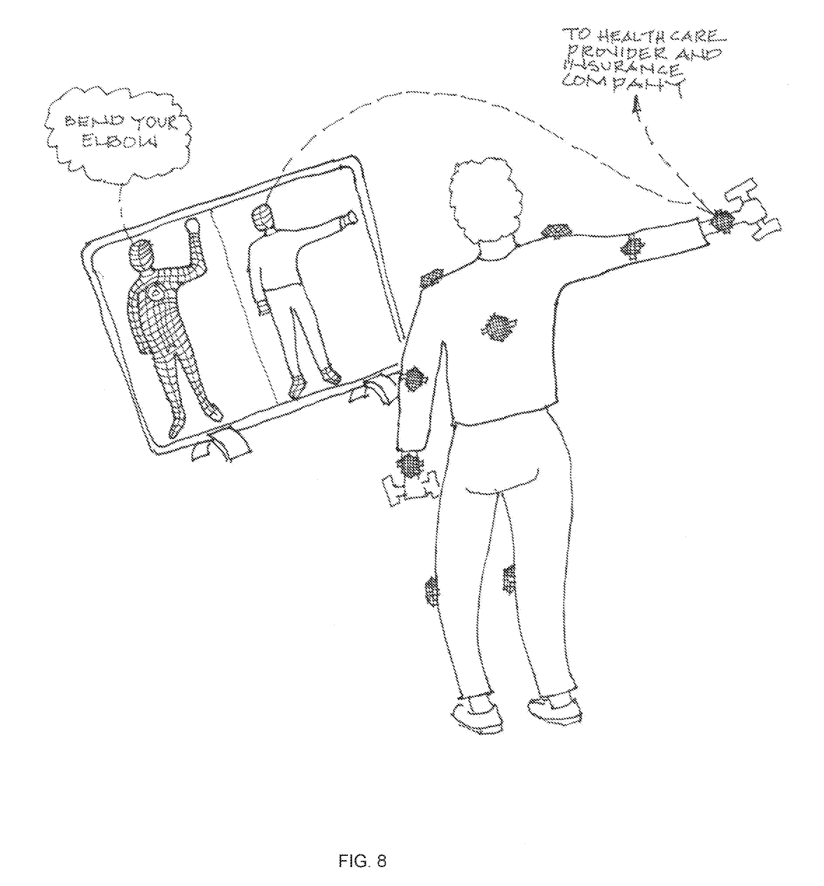

In a preferred embodiment, one or more sensors are attached to a user's body for determining not only whether the user is exercising but also to determine whether he is in fact exercising correctly and, consequently, a second avatar can be generated. See FIG. 8. Moreover, the present inventors have discovered that body sensors, if properly installed and calibrated can be used to generate a video of the user's body motion during exercise. This information could be used for various purposes. For example, the moving image of the user can be played to the user side by side with the avatar (see FIG. 8) so that the user can compare his movement to the movement of the avatar during a particular exercise. In one embodiment, the sensor output can be used to trigger the process for generating images to the user. The sensor output can also be used to determine how fast or accurately the user is moving, and to use this information to synchronize the movement of the avatar to the actual movement of the user in real time.

In this embodiment of the invention, prior to starting an exercise regime, the user attaches one or more sensors to his body (see FIG. 8). In general, at least some sensors are positioned at or near body joints, such as elbows or knees. While sensors are available that could be installed at some of the joints and other additional or alternative critical locations, the inventors have discovered that if appropriate sensors are used, it is sufficient if these sensors are only placed at specific locations of the body. More specifically, it is sufficient to place a sensor device on each limb and one near the stomach. Each sensor device is connected directly or indirectly to a standard control device such as a desktop, a smart phone or the like and the plurality of sensors is commonly calibrated. For example, this connection may be accomplished using a Bluetooth protocol. The user can then start exercising. As discussed above, each exercise session includes several exercises determined by a regime prescribed to the user.

Programming in the system tells the patient in advance which exercise to do next, and provides other instructions, such as a brief description, important DO's and DON'T's, the number repetitions, and so on. The system can receive generated information for display, take input from the patient, or self-adjust based on the system's absorbed data regard patient exercises and the success rate of each or portions of each. Next, a stylized image of the user may be generated for display on a screen and presented to the user during the exercise showing in real time accurate images of the user and his body so that the user can determine whether he is exercising correctly or not.

In one embodiment, each (or at least one) sensor is coupled to the control device. Then software provided in the control device calculates a vector position of all human body articulations. The sensor device which is referred to as an I.M.U. (Inertial Measurement Unit) in actuality contains three types of sensors each with three axis of distinction (that can be referred to as being in an X, Y and Z orientation). This type of sensor is sometimes termed a 9DOF (or 9 degrees of freedom; 3×3) sensor. The sensor device of the present invention may have a plurality of internal sensors.

The first of these internal sensors is a magnetometer which has the capacity to sense earth's geomagnetic field and used in determining the device's position in space (this is a 3 dimensional representation having three axis).

The second sensor is an accelerometer which determines change in velocity providing information about the real time motion of the device, and therefore the respective limb.

Lastly, a gyroscope is provided which can determine the pitch, roll and yaw in both final placement of a body through a rotational representation.

All three of these sensors rely on a MEM (Micro Electro-Mechanical) technology and are coupled to an internal microprocessing unit within the sensor device.

The data from each sensor is processed by a microprocessor using an algorithm which transforms the data into a packet that represents the relationship between all axes and the sensors' position in space. The sensor device could be a Bosch BM0055 I.M.U. or equivalent. This final data packet is what is described as ‘Fused Data’ or ‘Fusion’ for short.

The packet of data that describes this very complex vector is known as a ‘Quaternion’. From ‘Wiki’: “In modern mathematical language, quaternions form a four-dimensional associative formed division algebra over the real numbers, and therefore also a domain”. The Quanternion defines the position of each device in real time in space.

In summary, the present invention includes means, such as but not limited to an app and/or configured into a processor, for sorting and sequencing exercises which may have been prescribed by a professional, where the exercises are organized to provide a sequence based on practicality and/or patient capability. Information regarding the patient's experiences with the exercises is shared with the professional so that the regimen can be further customized by the professional.

The exercises may be replicated for presentation to a patient by an avatar on a screen, where the avatar provides the patient with guidance for movement and which a patient may follow in concurrence with the display, the display being that of the avatar in the motions of the exercise, including repetitions and hold times. The avatar is intended to display the preferred (or even potentially optimal) approach relative to that patient. The avatar preferably shows all movements for a particular exercise so as to allow the patient to replicate the movements. The movements are intended to be indicative of the professional's view of how the patient might best be served. Patients can alter the speed of each component of the exercise, and individual hold times. Patients an also pause and restart the sequence.

Further, the present invention is implementable by a patient in at least two modes—a “Learn” mode and a “Play” mode. In the Learn mode a patient sees the avatar perform exercises, potentially as a continuous loop with additional audio instructions. For example, the patient can learn the exercises before trying to perform them. If necessary, the patient can review this Learn mode at any time. In one embodiment, using two fingers on an on-screen image, the patient can manipulate the presentation and, as an example, change the views of the avatar to different angles in order to better understand the position that the patient's body is supposed to be in when actually performing the exercise. Play mode is run by the patient when ready to follow and perform the actual exercises.

In one embodiment, when the system of the present invention is operated in “Play” mode, data are collected on the patient's usage, and used in an analysis, at least in part based on a comparison to the approach shown in the avatar. Feedback may be available to both the patient and the professional.

The present invention further includes a search and selection wizard for search and selection of exercises from a library of exercises included within a database. Search and selection may be facilitated by using onscreen filter categories, search functions, and thumbnail images of each exercise, Filter categories work with specific definitions set up in the database for each exercise in categories which are pertinent to physical therapy concerns. As categories are checked off, the wizard automatically culls down the number of exercises that are viewable to those selected. The professional can customize the range of categories to match the areas of their practice and can select either individual exercises from standard library, or from a professional's customized library, or programs of already grouped exercises from the program library.

Exercise set up can include the following:

1. Parameters set by the professional specifically for each patient, such as on the professional's website.

2. Selected parameters may include:
a. Number of repetitions of each movement.
b. Duration of time to hold certain positions.
c. Number of sets and length of time in between sets.
d. Side of body.
e. Weights and resistance bands.
f. Number of times per day performed.

3. The professional can alter the exercise, for example by changing the angle of a leg or arm of an exercise. This may be done based on data received relative to the patient's actions and usage,

4. An implemented algorithm, possibly implementable based on user actions in a graphical user interface, enables the professional to select back-up exercises that are substituted, if a patient indicates that an exercise is too easy or difficult.

Exercising can be scheduled for the patient, such as based on a professional's guidance. The professional can assign an exercise on a daily, weekly, or monthly calendar. The present invention includes implementing an algorithm for which group of exercises to show the patient based on the number of times assigned per day coupled with the number of times watched per day. So if a patient is assigned three exercises to be done once a day and two other exercises to be performed twice a day once the first group has been watched and implemented it will no longer be displayed for the patient that day. This assignment can be for an extended time, such as a multi-month program and adjustable based on patient usage.

In one embodiment, the system of the present invention includes a plurality of sensors, each of which is attachable to a patient at a different place on the patient's body. These may be attachable in a temporary way, such as by a rubber band-like attachment or a sticky pad or could be attached to a watch or similar device. Attachment may be made at or near a plurality of joints, such as wrists, knees, shoulders, and so on. Each sensor's location may be identifiable and each sensor includes the ability to communicate its location and/or movement to a core processor (which could be a distributed processor) and each sensor sends the core processor information regarding its location and/or movement, which the core processor can use to reflect on the patient's movement. In one example, the core processor receives data at regular intervals, such as per second or more frequently, from each sensor, where the data includes changes in position from the previous second.

Each sensor has connectivity to a base, where the connectivity is preferably wireless, although it could alternatively be wired. In one embodiment, the base also has connectivity to the delivered avatars, at least for the purpose of time synchronizing patient movement with avatar movement.

In addition, the data collected from each sensor, as well as the data collected from the collection of sensors, are compared to the standard used for the avatar display (or some equivalent). Deviations can be identified by particular exercise or particular movement or, for example, a limitation to the patient's range of motion.

The present invention includes a series of wearable inertial measurement units (see FIG. 8) transmitting data, such as to a smartphone, which can serve the purpose of the “base” as described above. The present invention includes apparatus, such as but not limited to a processor, configured to analyze the data, compare it to data of commonly prescribed exercises, and determine if the number of repetitions of the exercise has been completed, or if they were attempted but not completed correctly, or not attempted at all.

Movement is determined based on analysis of data gatherers in each sensor. Each sensor includes at least one three-axis gyroscope, three-axis accelerometer, and a three-axis magnetometer, each of which determines its movement for transmission to the processor.

The process involves a very sophisticated mathematical formula that uses data derived from a set of 3 dimensional movements. In particular and in our case we are looking at 3 dimensional rotational data which gives us what we need to calculate a relative point in space and how it moves.

In the preferred embodiment, the processor gathers the data, determines movement from the gathered data, and synchronizes the determined movement to the avatar display (or an equivalent) so as to determine deviation from the desired approach and where the patient successfully performed the exercises. The results from the sensors need to be filtered, where such filtering is done in the form of mathematical equations. This filtering, referred to as “fusion”, requires culmination of all the portions of calculating movements and variations from the desired standard. As a part of that filtering, a Kalman filter (also known as a ‘linear quadratic estimation’) may be applied that takes care of the deleterious drift that accompanies gyroscopes.

In one embodiment, once a descriptive vector is determined it is passed along to a device named ‘Simblee’ (by the RFDigital corporation), or to an equivalent device serving the same or similar purpose(s). This is a low energy, Bluetooth device which also contains its own microprocessor. While Simblee and Bluetooth are used descriptively herein, they are merely example embodiments and comparable wireless technologies and other than iOS implementations may be used in the context of the present invention. The Bluetooth microprocessor role is to poll the I.M.U. for its data and then wirelessly pass the pertinent information to the control device which then imposes the instruction that represents a visual motion or at least indicators of positive or negative motion of a human body model. The software contained in this device which may be pre-programmed does the ‘housekeeping’ for the I.M.U. and the later transmission of the fused data.

Software for the Simblee is in a modified Arduino code proprietary to RF digital products (an enhanced version of the C++ language).

Also the software that interprets how the above data which influences the U.I. (User Interface) visual human body model and its articulation for the iOS's response is also included.

The U.I. shows numbers of repetitions completed, the level to which a motion was executed and to what extent a portion of a body maintains a proper torque to an articulation.

An ultimate goal of the device is to take a known and correct articulation of a body's movement and compare this absolute with the movement of a participant and correlate the two actions as how they compare, correcting a real time motion as it is determined to be less than what is expected of this participant.

The system includes a means of prescribing a particular motion, through a system such as a cloud-based program, and the ability to record the motion executed for later analysis automatically or by an individual familiar with proper and improper executions of these exercises.

Use of the sensor device is shown in FIG. 8. In the figure, a user is shown with what may appear to be a watch together with additional sensors. While a watch may embed a sensor, each sensor may be stand-alone attached to the user or may be embedded in another object, such as but not limited to a watch, a bracelet, a ring, or an article of clothing. In addition, the figure shows a screen device with avatars performing the exercises. While shown with avatars, the screen device need not show the avatar, such as when a user is experienced with the exercise. The screen device, however, is in communication with the sensor devices and a user can control activity by touching (and/or verbally communicating) with the screen device, such as to start or stop exercises. By touching or otherwise communicating with the screen device, the screen device can communicate real time or near real time program changes to the sensors attached to the user.

In the Simblee embodiment described herein as an example, the elements of the device and their functions are preferably as follows (of course, comparable alternatives could also be used):
R1—resistor for 12C serial data
R2—resistor for 12C serial data
R3—resistor for momentary contact switch (SW2)
R4—for Reset for I.M.U designated: U1 (reset unused at this time)
C1—capacitor for the Simblee
C2—(U3) output filter capacitor
C3—(U3) input filter capacitor
C4—capacitor for Xtal in
C5—capacitor for Xtal out
C6—capacitor for I.M.U.
C7—capacitor for I.M.U.
C8—capacitor for I.M.U.
C9—capacitor for I.M.U.
SW1—Power Switch
SW2—Momentary Contact Switch
Note: P0.05 pin on Simblee=12C SGL (clock) P0.06 pin on Simblee=12C SDA (data)

### Pin 17 on I.M.U.=GND

In summary, the present invention encompasses a creation mode, a data capture mode, an implementation mode, and a playback mode, wherein the modes are not necessarily separate but are described as such herein for purposes of clarity. In the creation mode, the present invention is directed to a system that allows a professional (therapist, physician, etc.) to select exercises, customize the exercises to a patient's needs, and arrange for them to be displayed to an end user, such as a patient or other client. The system includes ability for selection and displaying exercises, and includes an algorithm for sequencing the exercise, such as based on body position, equipment used, or type of exercise, etc. Once the system sequences the exercises, a professional may alter the system's initial selection, such as altering through use of a graphical user interface. The display of exercises to a user can include the ability to highlight muscles so as to educate the patient as to what/where they should be feeling as they exercise correctly.

The present invention uses a game engine, but to save time, the whole game engine isn't necessarily downloaded; only those parts that are needed for each session may be downloaded. The game engine data is not necessarily streamed. So users can access the data after it's been downloaded, even when the user is not connected to wifi. Statistics and exercises are updated once a connection is reestablished. Downloading only some of the game engine, and not streaming any of it, shortens the time required to download a patient's routine, saves data, and also allows for access to the routine offline.

Images are identified and selected by the game engine of the present invention.

Animations may be stackable one on another. This “stacked animation” makes it possible to key frame animate, that is, animate from scratch, a single joint animation and put ‘noise’ on top of it so it will look more lifelike. Motion capture is primarily used in the present invention. “Stacked animation” is a technique introduced in the present invention in which key frame animation is added into motion capture animation as needed. “Noise” is basically extra movement in order to make movement more lifelike. Consequently, the present invention combines key frame animation with motion capture animation for use in a game engine.

The system of the present invention further includes a calendar feature for automatically changing the exercise regimen for the patient, such as to align with that programmed by the professional; that is, the program can change by or within the week or the month, or the whole protocol can be adjusted based on questions answered by the patient and by use of the system. The system of the present invention also allows the patient to skip exercises that they don't like, and, when skipped, the skipping is reported to the professional. The professional can subsequently change the patient's program based on actionable data, to improve compliance.

The data capture mode is based on data collected using wearable sensors (“wearables”). The wearables of the present invention include IMUs which collect and transmit data to a receiving device, such as but not limited to a smartphone, wherein the device accepts data from the wearables, compares the data to that of commonly prescribed exercises, determines if the number of receptions of the exercise has been completed, or if exercises were attempted but not completed correctly, or not attempted at all.

Further, the system of the present invention captures data regarding the exerciser, in part for further analysis and refinement of exercises. Data are captured through the deployed sensors and the processor of the present invention uses the captured data, in comparison to the prescribed exercises, to determine compliance and non-compliance and to analogize to potential limitations the exerciser may have. Once the data are analyzed, the processor of the present invention can suggest to a professional, or directly implement, changes to the regimen for the exercising patient. For example, if the exercise protocol requires holding a particular position for ten seconds and the patient demonstrates (once or over a period of days) that the position can only be held for five seconds, the processor can assess why the patient cannot hold the position and adjust the regimen or suggest alternatives. In addition, the non-compliance is reportable to the professional who can also adjust the regimen. In a sense, the regimen may be adjusted so that the patient is not advance-notified of the adjustment. That is, because of such adjustments, or because of day-to-day changes in the regimen, or other reasons, the patient's regimen may differ from day-to-day (or session-to-session), requiring the patient to stay focused on the avatar when exercising.

Moreover, and very importantly, the wearables can be used collectively as a very portable, camera-less motion capture system. Physical therapists and people in the entertainment industry, among others, can use the wearables to capture motion data for use in medical and entertainment purposes. Professionals, such as but not limited to physical therapists and personal trainers (and others) use these wearables to create avatars specific to patients so that the patients and clients can follow along.

In the implementation mode, the system of the present invention may include the ability to alter or adjust exercise speed, such as but not limited to during playback, including speed for each exercise and speed during the display of an exercise. In other words, the system of the present invention provides for dynamic feedback on results of a program, customized to a patient, where the feedback can be provided element-by-element (e.g., one portion of an exercise to the next portion), from numerous perspectives, and time adjusted, with requisite pauses of finite duration built in. The system of the present invention may parse an exercise (either as provided as the model, or as recorded of the patient performing the exercise) into different portions. Through a system of flagging each portion of an exercise (e.g., the entrance, the middle, the exit), a user can have the speed customized and a customized hold time can be programmed into the playback. This means that each physical therapist can customize the speed that they want their patients to perform each part of the exercise so as to maximize customization. This includes introduction of hold times for select positions.

The present invention allows for introducing specific hold times for each exercise. By extending the hold time (such as via an online input) we can increase or decrease the hold, selectable possibly either by the professional, the patient, or both. With this technique we are able to show a lifelike motion in a hold. This means that when the avatar is holding in a position, it's not just stopping the action, it is actually and intentionally delaying the next motion.

In the playback mode of the present invention, a motion capture file is initially divided in segments. Seamless loops are created and used for playback. The playback speed of the animation can be varied without the feeling that the playback is speeded up or slowed down. We can specify a baseline playback speed, adjusted based on, for example, a patient's attributes, such as age. Older people tend to move slower. The baseline speed is further adjustable by a professional based on other factors (e.g., known physical limitations of the patient). The patient can potentially further control the playback speed in real time as needed. Also, because the data collected by the sensors allow the system of the present invention to model the actual patient movements in three dimensions, and the system of the present invention incorporates the ability for three dimensional modeling, the orientation of the patient during playback can be adjusted as well, such as by rotating the presentation to show a side view or a top-down view. This rotation and orientation may be controllable, at least in part, by the viewer.

The present invention implements both a motion creation tool and a playback tool, together controlled by a processor (subject to any user control) in the present invention. Motion capture uses motion from an actor so as to show lifelike motion. The motions, initially loaded into the motion capture tool, result in a usable file which can be controlled by the processor of the present invention (e.g., where to loop, where the hold times should be, the different parts of each exercise, etc.). The newly-created motion file is then applied to the different exercises.

The 3D geometry and motion data is recorded from an actor (person) actually moving through the exercise being recorded, while wearing devices transmitting data to receivers. This is referred to as motion capture. A 3D modeling software program is then used to create a digital 3D model. To this 3D model, visual characteristics such as color, texture, etc., are added, which develops the imagery of the model, using a game engine. In addition, the game engine enables controls of other aspects of the display of the model on the viewing devices. The game engine is used by us, not only to add visual characteristics (usual), but to also enable the exercises to be flagged at different points and each part played at different speeds within each exercise.

The playback tool is usable to playback the exercises subsequent to adjustment in the 3D space. The file for playback is arranged so that it is controllable by a user, such as controllable in time or presentation orientation. Unlike video, it can be interacted with. One can rotate the model, zoom in/out and the exercise will just be playing. The playback tool provides a 3D engine, which is controllable on a variety of playback devices, including mobile devices. As an alternative, the exercises could be played back as a streamed video, but that would always require at least a wifi connection fast enough to play back accurately.

Consequently, the present invention serves as a closed system, in which the professionals create exercises that they want their patients to follow, and patients wear the wearables, thereby serving to confirm that they in fact performed the exercises. Patients receive feedback about the correctness of their performance and, if they want, see an avatar that they generate by using the wearables.

Healthcare professionals and personal trainers, providers (doctors), insurance companies, etc., have the ability with the present invention to monitor patient compliance and provide patients much needed feedback (providable automatically and separately by the professional) on the correctness of their movements. Patients can use the present invention in similar ways. The entertainment industry can use the wearables for motion capture animation, at a fraction of the cost of traditional motion capture approaches, such as involving expensive cameras, making the system of the present invention accessible and more affordable.

1. A system for generating a customized audio/video program of sequenced exercises for a specific user in response to instructions defining a regimen of exercises for the user, said system comprising:
a server in communication with a database and a practitioner interface, said server receiving a regimen and user specific data from said practitioner interface, said database including a movement library defining each exercise of said regimen as a plurality of movements of body parts;
a game engine connected to said library and adapted to generate a video track for each said exercise based on said movement library, each said track displaying an avatar with avatar body parts moving to simulate an exercise, said engine configured to collect and deliver to a data store usage playback data relative to each exercise;
an automated director configured to generate a customized exercise program for said user by compiling said video tracks and corresponding audio clips as a sequence of exercises, said sequence calculated based on said user specific data in combination with a person's exercise-to-exercise re-positioning; and
said practitioner interface configured to receive said usage playback data and for facilitating a practitioner's adjusting said regimen;
wherein said user specific data include physical limitations of the user.

2. The system of claim 1 further comprising an avatar module configured to select said avatar from an avatar library; and said game engine and said director cooperating to display said avatar performing said exercises in line with said limitations.

3. The system of claim 1, wherein said director further calculates said sequence by grouping said exercises based on at least one of an end position of the user during each exercise, equipment change required for each exercise, and the user's resting body part during each exercise.

4. The system of claim 1, wherein said user specific data further indicates physical characteristics of the user and the movement of the avatar in said program is changed by said server in accordance with said physical characteristics of the user.

5. The system of claim 1, further including a plurality of sensors disposed on the users body for collecting user movement data.

6. The system of claim 5, wherein each said sensor is comprised of at least one accelerometer, at least one gyroscope, and at least one magnetometer.

7. The system of claim 5, wherein said user movement data is delivered to said practitioner interface for further facilitating adjusting said regimen.

8. The system of claim 5, wherein said user movement data is time synchronized to said usage playback data, and differences are made available to at least one of a user interface and said practitioner interface.

9. The system of claim 8, wherein said differences are used as input to said practitioner interface for use in adjusting said regime.

10. The system of claim 8, wherein said server analyzes said differences to identify specific body movements of difference.

11. A method for a processor-based server, comprising an automated director and a game engine, to generate and deliver a customized exercise program comprising moving avatars to a user to prompt the user to perform an exercise session, said program including a video component, said method comprising the steps of:
receiving a regimen of exercises for a user from a practitioner interface;
using an automated director, arranging said exercises into a customized sequence based on user physical limitations and a person's exercise-to-exercise re-positioning;
retrieving from an avatar library a plurality of avatar body part movements corresponding to said exercises;
using a game engine, creating video images of said avatar performing said exercises based on information from an avatar library,
said director compiling said images based on said customized sequence; and
presenting said program to the user on a user device.

12. The method of claim 11 wherein said program includes a first section including text or visual information describing the regimen, a second section with a demonstration of one or more of the exercises, and a third section consisting of the avatar performing one or more exercises.

13. The method of claim 11, further including the steps of collecting movement data and adjusting said program based on difference from said presented program.

14. The method of claim 13, wherein a plurality of physical monitors are disposed on a user's body collect said movement data; each said monitor comprising at least one accelerometer, at least one gyroscope, and at least one magnetometer.

15. The method of claim 14, wherein said difference is delivered to said practitioner interface for further facilitating adjusting said regimen.

16. The method of claim 14, wherein said user movement data is time synchronized with said presented program for analysis of differences.

17. The method of claim 16, wherein difference is made available to at least one of said user interface and said practitioner interface.

18. The method of claim 16, wherein said difference is used as input to adjusting the exercise sequence in said sequence.

19. The method of claim 16, wherein said difference is used as input to adjusting individual exercises in said sequence.

20. The method of claim 16, wherein said server analyzes said difference to identify specific body movement limitations and adjusts said program accordingly.
